(19) United States
(12) Patent Application Publication
(10) Pub. No.: US 2009/0254139 A1
(43) Pub. Date: Oct. 8, 2009

(54) IMPLANTABLE MEDICAL DEVICE WITH OPTIMIZATION PROCEDURE

(75) Inventor: Anders Bjorling, Solna (SE)

Correspondence Address: SCHIFF HARDIN, LLP;PATENT DEPARTMENT, 233 S. Wacker Drive-Suite 6600, CHICAGO, IL, 60606-6473, US

(73) Assignee: ST. JUDE MEDICAL AB, Jarfalla (SE)

(21) Appl. No.: 12/296,979

(22) PCT Filed: Apr. 13, 2006
(86) PCT No.: PCT/SE2006/000441

§ 371 Date: Jun. 17, 2009

(51) Int. Cl. A61N 1/365

(52) U.S. Cl. 607/17

(57) ABSTRACT

In an implantable medical device and a method for the operation thereof, acoustic energy is sensed in a subject in whom the device is implanted, and signals indicative of heart sounds of the heart of the patient are produced over predetermined periods of a cardiac cycle, during successive cardiac cycles. A signal corresponding to the second heart sound (S2) is extracted from the sensed signal, and the respective durations of successive second heart sound signals are determined. An optimization procedure is implemented that includes controlling delivery of pacing pulses based on the determined durations of successive second heart sounds, to determined a combination of stimulation intervals, including at least the AV interval and the VV interval, that causes a substantially synchronized closure of the aortic and pulmonary valves.

### TECHNICAL FIELD

The present invention generally relates to implantable medical devices, such as cardiac pacemakers and implantable cardioverter/defibrillators, and in particular to a method, an implantable medical device, a computer program product and a computer readable medium for optimizing therapy by minimizing the systolic phase of a heart of a patient using detected heart sounds.

### BACKGROUND OF THE INVENTION

Auscultation is an important diagnostic method for obtaining information of the heart sounds, which is well established as diagnostic information of the cardiac function. The sounds are often described as S1-S4. During the working cycle of the heart mechanical vibrations are produced in the heart muscle and the major blood vessels. Acceleration and retardation of tissue cause the vibrations when kinetic energy is transformed to sound energy, e.g. at valve closing. Vibrations can also arise from turbulent blood flow, e.g. at stenosis and regurgitation. These vibrations may be listened to using a stethoscope or registered electronically using phonocardiography, i.e. graphical registration of the heart sounds by means of a heart microphone placed on the skin of the patient's thorax. Auscultation using a stethoscope is, to a large extent, built on practical experience and long practice since the technique is based on the doctor's interpretations of the hearing impressions of heart sounds. When applying phonocardiography, as mentioned above, a heart microphone is placed on the skin of the patient's thorax. In other words, it may be cumbersome and time-consuming to obtain knowledge of the heart sounds and the mechanical energy during the heart cycle using these manual or partly manual methods and, in addition, the obtained knowledge of the heart sounds may be inexact due to the fact that the knowledge is, at least to some extent, subjective.

The first tone S1 coincides with closure of the mitral and tricuspid valves at the beginning of systole. Under certain circumstances, the first tone S1 can be split into two components. An abnormally loud S1 may be found in conditions associated with increased cardiac output (e.g. fewer, exercise, hyperthyroidism, and anemia), tachycardia and left ventricular hypertrophy. A loud S1 is also characteristically heard with mitral stenosis and when the P-R interval of the ECG is short. An abnormally soft S1 may be heard with mitral regurgitation, heart failure and first degree A-V block (prolonged P-R interval). A broad or split S1 is frequently heard along the left lower sternal border. It is a rather normal finding, but a prominent widely split S1 may be associated with right bundle branch block (RBBB). Beat-to-beat variation in the loudness of S1 may occur in atrial fibrillation and third degree A-V block.

The second heart sound S2 coincides with closure of the aortic and pulmonary valves at the end of systole. S2 is normally split into two components (aortic and pulmonary valves at the end of systole) during inspiration. Splitting of S2 in expiration is abnormal. An abnormally loud S2 is commonly associated with systemic and pulmonary hypertension. A soft S2 may be heard in the later stages of aortic or pulmonary stenosis. Reversed S2 splitting (S2 split in expiration-single sound in inspiration) may be heard in some cases of aortic stenosis but is also common in left bundle branch block (LBBB). Wide (persistent) S2 splitting (S2 split during both inspiration and expiration) is associated with right bundle branch block, pulmonary stenosis, pulmonary hypertension, or atrial septal defect.

The third heart sound S3 coincides with rapid ventricular filling in early diastole. The third heart sound S3 may be found normally in children and adolescents. It is considered abnormal over the age of 40 and is associated with conditions in which the ventricular contractile function is depressed (e.g. CHF and cardiomyopathy). It also occurs in those conditions associated with volume overloading and dilation of the ventricles during diastole (e.g. mitral/tricuspid regurgitation or ventricular septal defect). S3 may be heard in the absence of heart disease in conditions associated with increased cardiac output (e.g. fever, anemia, and hyperthyroidism).

The fourth heart sound S4 coincides with atrial contraction in late diastole. S4 is associated with conditions where the ventricles have lost their compliance and have become “stiff”. S4 may be heard during acute myocardial infarction. It is commonly heard in conditions associated with hypertrophy of the ventricles (e.g. systemic or pulmonary hypertension, aortic or pulmonary stenosis, and some cases of cardiomyopathy). The fourth heart sound S4 may also be heard in patients suffering from CHF.

Thus, the systolic and diastolic heart functions are reflected in the heart sound and knowledge of the heart sounds may, for example, be used for diagnosis/monitoring and controlling pacing therapy of patients. This knowledge may hence be used to optimize a stimulation therapy and to verify that the stimulation output evokes a desired response in a selected region of the heart. One of the major objectives of CRT devices (Cardiac Resynchronization Therapy devices) is to increase the length of the diastolic phase by coordinating the left and right ventricles' contraction patterns. In patients receiving such a CRT device (Cardiac Resynchronization Therapy device), the initial health status can be very poor and it is therefore utterly important to optimize programmable parameters such as AV interval (or delay) and VV interval (or delay), not only at implant but also as time progresses and the heart is remodeled. Consequently, it would be beneficial if signals related to the heart sounds could be collected and used for controlling/optimizing pacing therapy in an automated manner. For the patient, an automatic AV and VV interval optimization algorithm would mean fewer visits to the hospital and improved care. A pacemaker algorithm that is able to optimize the parameters itself would also be beneficial for the physician. Today, pacing intervals such as VV intervals or intervals are often optimized on basis of echocardiographic studies performed to determine the settings resulting in the best hemodynamic response. After evaluation of hemodynamic effect of varying combinations of pacing intervals, a physician must manually select and program the desired parameters and assume that the optimal setting of the device remain unchanged, at least until a potential subsequent re-optimization visit. This procedure is thus time consuming and is often performed by someone else than the implanter. If the device could perform this automatically, fewer steps would thus need to be executed at the hospital and hospital resources can be freed.

Thus, when optimizing the parameters of a CRT, e.g. pacing intervals such as AV and/or VV interval or intervals, one usually tries to increase the diastolic filling time so that the heart is given more time to relax and to be filled with blood. Synchronizing atria and ventricles in order to minimize the systolic phase of the heart does this. For example, as discussed above, the second heart sound (S2) is caused by the closure of the aortic and pulmonary valves and in a patient with a dyssynchrony between the ventricles there may be a interval between these events. This may lead to a split S2 wave and it is possible to distinguish between two more or less overlapping S2 valves, namely A2 and P2, the closure of the aortic and the pulmonary valve, respectively. If the duration of S2 is small it means that the closure of the aortic and pulmonary valves occur simultaneously and that the ventricles are synchronized. The synchronization means an increased diastolic filling phase. Furthermore, as mentioned above, the first heart sound S1 is caused by the closure of the mitral and tricuspid valves and a short duration of S1 is an indication of the heart sides being synchronized and that the diastolic phase is long. Accordingly, in order to optimize the function of a CRT device, it would be beneficial if signals related to the heart sounds, in particular the first and second heart sounds, respectively, could be collected and used for controlling/optimizing pacing therapy in an automated manner such the length of the diastolic phase of the heart is increased.

The known technique presents a number of automated systems for controlling/optimizing stimulation therapy as, for example, U.S. Pat. No. 6,792,308 issued to Corbucci, which discloses an implantable medical device, such as a cardiac pacemaker, adapted to sense first and second heart sounds and to optimize the AV interval using the detected first and second heart sounds. In WO 2004/078257 issued to Chinchoy, a method and apparatus for monitoring left ventricular cardiac contractility and for optimizing a cardiac therapy based on left ventricular lateral wall acceleration are disclosed.

However, the prior art does not disclose a method for collecting information of the heart sounds and using the information to automatically controlling the stimulation therapy to increase the diastolic filling time.

### BRIEF DESCRIPTION OF THE INVENTION

Thus, an object of the present invention is to provide a method and an implantable medical device that are capable of automatically collecting information of the heart sounds and using the information to automatically controlling the stimulation therapy to increase the diastolic filling time so that the heart is given more time to relax and to be filled with blood.

Another object of the present invention is to provide a method and an implantable medical device that are capable of automatically collecting information of the heart sounds and using the information to automatically minimize the systolic phase of the heart.

A further object of the present invention is to provide a method and an implantable medical device that are capable of automatically collecting information of the heart sounds and using the information to automatically control a stimulation interval combination including the AV interval (or AV delay) and the VV interval (or VV delay) to obtain a substantially synchronized closure of the aortic and pulmonary valves.

Yet another object of the present invention is to provide a method and an implantable medical device that are capable of automatically collecting information of the heart sounds and using the information to automatically controlling a stimulation interval combination including the VV interval to obtain a substantially synchronized closure of the mitral and tricuspid valves.

A further object of the present invention is to provide a method and an implantable medical device that are capable of automatically collecting information of the heart sounds and using the information to automatically controlling a stimulation interval combination including the AV interval (or delay), which may include the AV interval, the AR interval (or delay), the PR interval (or delay) or the PV interval (or delay), and/or the VV interval (or delay), which may include the VV interval, to obtain a substantially synchronized closure of the aortic and pulmonary valves and a substantially synchronized closure of the mitral and tricuspid valves, respectively.

These and other objects are achieved according to the present invention by providing a method, an implantable medical device, a computer program product and a computer readable medium having the features defined in the independent claim. Preferable embodiments of the invention are characterised by the dependent claims.

According to an aspect of the present invention, an implantable medical device including a pulse generator adapted to produce cardiac stimulating pacing pulses is provided. The device is connectable to at least one lead comprising electrodes for delivering the pulses to cardiac tissue in at least one ventricle of a heart of a patient and comprises a signal processing circuit adapted to extract a signal corresponding to a second heart sound (S2) from a sensed signal, the signal being received from an acoustic sensor adapted to sense an acoustic energy and to produce signals indicative of second heart sounds of the heart of the patient over predetermined periods of a cardiac cycle during successive cardiac cycles, and to determine a duration of successive second heart sound signals. Furthermore, the device comprises a controller adapted to perform an optimization procedure, wherein a delivery of the pacing pulses is controlled based on determined durations of successive heart sound signals to identify a combination of stimulation intervals including an AV interval and a VV interval that causes a substantially synchronized closure of the aortic and pulmonary valves.

According to a second aspect of the present invention, there is provided a method for operating an implantable medical device to obtain substantially synchronized closure of the aortic and pulmonary valves, the device including a pulse generator adapted to produce cardiac stimulating pacing pulses and being connectable to at least one lead comprising electrodes for delivering the pulses to cardiac tissue. The method comprises the steps of: sensing an acoustic energy of the heart of the patient; producing signals indicative of heart sounds of the heart of the patient over predetermined periods of a cardiac cycle during successive cardiac cycles; extracting a signal corresponding to a second heart sound (S2) from a sensed signal; determining durations of successive second heart sound signals; and performing an optimization procedure, the optimization procedure comprising the step of controlling the pacing pulses based on the determined durations of successive second heart sounds to determine a combination of stimulation intervals including an AV interval and a VV interval that causes a substantially synchronized closure of the aortic and pulmonary valves.

According to a third aspect of the present invention, a signal corresponding to a first heart sound (S1) is also extracted from a sensed signal; durations of successive first heart sound signals are determined; and the pacing pulses are iteratively controlled based on the determined durations of successive first heart sounds to identify stimulation interval including a VV interval that causes a substantially synchronized closure of the mitral and tricuspid valves.

According to a fourth aspect of the present invention, there is provided a computer program product, which when executed on a computer, performs steps in accordance with the second and/or third aspect of the present invention.

According to a further aspect of the present invention, there is provided a computer readable medium comprising instructions for bringing a computer to perform steps of a method according to the second and/or third aspect of the present invention.

Thus, the invention is based on the idea of, in an implantable medical device, collecting or obtaining information of the heart sounds, which carry valuable information of the workload and status of the heart, and using this information to automatically controlling the stimulation therapy of the implantable medical device, such as a CRT device, to increase the diastolic filling time so that the heart is given more time to relax and to be filled with blood. In particular, the invention is based on the insight that this can be achieved by synchronizing the closure of the aortic and pulmonary valves by interatively controlling an AV interval and a VV interval and/or by synchronizing the closure of the mitral and tricuspid valves by iteratively controlling a VV interval.

This invention provides several advantages. For example, the length of the diastolic phase can be increased since contraction patterns of the left and right ventricles' are coordinated. Thereby, the systolic phase of the heart is minimized, which, in turn, means a longer “resting” period of the heart. The stimulation parameters of the device, such as AV interval and VV interval, may be continuously and automatically adjusted not only at implant but also as time progresses and the heart is remodeled. Furthermore, the automatic stimulation parameter optimization, e.g. the AV and VV interval optimization algorithm, would mean fewer visits to the hospital and improved care. A pacemaker algorithm that is able to optimize the parameters itself is also of great benefit for the physician. Today, pacing intervals such as VV intervals are often optimized on basis of echocardiographic studies performed to determine the settings resulting in the best hemodynamic response. After evaluation of hemodynamic effect of varying combinations of pacing intervals, a physician must manually select and program the desired parameters and assume that the optimal setting of the device remain unchanged until a subsequent potential re-optimization visit. This procedure is thus time consuming and is often performed by someone else than the implanter. Consequently, since the device is able to do perform this automatically, fewer steps is hence needed at the hospital and hospital resources can be freed.

Another advantage is that the optimization of the stimulation parameters can adapt to changing conditions of a heart of a patient in a fast and reliable way since intrinsic information of the heart, i.e. the heart sounds, is used as input information, in turn, leading to a better security for the patients in different situations. The results is also accurate due to the facts that the systolic and diastolic heart functions are reflected in the heart sound, and that the heart sounds and their relations thus carry information of the workload and status of the heart.

The fact that the heart sounds are obtained by means of an implantable medical device connectable to an acoustic sensor that senses sounds or vibrations inside or outside the heart also contributes to higher degree of accuracy and reliability.

According to one embodiment of the present invention, the stimulation intervals are controlled such that the durations of the second heart sound signals and or the first heart sound signals are brought to be within a predetermined range of durations. This predetermined range may be programmable, which entails that the range can be adjusted for different patients or adjusted in response to changing conditions of a patient and thus the AV interval and VV interval can be optimized with a high degree of accuracy. Alternatively, the stimulation intervals are controlled such that the durations of said second heart sound signals are minimized. Thereby, an AV interval and a VV interval that synchronizes a closure of the aortic and pulmonary valve can be obtained in an accurate and automated way. In addition, the VV interval can be controlled such that the durations of said first heart sound signals are brought to be within a predetermined range of durations, which range also may be programmable, or are minimized. Thereby, a VV interval that synchronizes a closure of the mitral and tricuspid valve can be obtained in an accurate and automated way.

In a further embodiment, a sum of a duration of a first heart sound and a duration of a second heart sound for successive cardiac cycles is calculated; and the pacing pulses is controlled based on the calculated sums of durations to identify a stimulation interval combination, e.g. a combination of AV interval and VV interval that causes a substantially synchronized closure of the mitral and tricuspid valves and/or a substantially synchronized closure of the aortic and pulmonary valves, respectively. Accordingly, the length of the diastolic phase can be increased since the contraction patterns of the left and right ventricles' are coordinated and, thus, the systolic phase of the heart can be minimized.

According to one embodiment, the stimulation interval combinations are controlled such that the said sums of a first heart sound and second heart sound are brought to be within a predetermined range of duration sums, which range also may be programmable, or such that the sums of a first heart sound and second heart sound are minimized.

Alternatively, the sum of a duration of a first heart sound and a duration of a second heart sound for successive cardiac cycles can be calculated such that the first heart sound is weighted with a first weight and the second heart sound is weighted with a second weight.

In yet another embodiment, the duration of the period of time from start of the first heart sound to the end of the second heart sound for successive heart cycles is calculated, and the pacing pulses are controlled based on the calculated durations of the period of time from start of the first heart sound to the end of the second heart sound to identify a stimulation interval combination that minimizes the systolic phase. Alternatively, the stimulation interval combinations can be controlled such that the durations of the period of time from start of the first heart sound to the end of the second heart sound are within a predetermined range of durations, which range may be programmable, or such that the durations of the period of time from start of the first heart sound to the end of the second heart sound are minimized.

According to yet another embodiment of the present invention at least one bandpass filter is adapted to filter off frequency components of the acoustic signal outside a predetermined frequency range. The at least one bandpass filter may have a frequency range of 10 to 300 Hz. If two bandpass filters are used, a first filter may be adapted to cut out a predetermined frequency range corresponding to typical frequencies for the first heart sound, for example, 20-40 Hz, and a second bandpass filter may be adapted to cut out a predetermined frequency range corresponding to typical frequencies for the second heart sound, for example, 20-100 Hz, or 10-300 Hz. Furthermore, the determination of the durations can be based on a part of the filtered signal above a predetermined amplitude threshold, which threshold may be programmable. This further reduces the noise content of the signal. Alternatively, signals corresponding to the first heart sound (S1) and/or the second heart sound (S2) are extracted from a sensed signal by selecting a part of the sensed signal above a predetermined amplitude threshold, which threshold may be programmable. The duration may be calculated based on the selected part of the signal.

In another embodiment of the present invention, a breathing cycle of the patient is sensed, at least one predetermined point in the breathing cycle is identified, and the sensing sessions of the acoustic sensor is synchronized with the at least one predetermined point in said breathing cycle of the patient for successive breathing cycles. Thereby, the accuracy and efficiency of the optimization procedure can be further improved. This is mainly due to the fact that the S2 duration may depend not only on dyssynchrony, but also on where in the breathing cycle the measurement is made. During inspiration, negative intrathoracic pressure causes increased blood to return into the right side of the heart. The increased blood volume in the right ventricle causes the pulmonic valve to stay open longer during ventricular systole. This causes an increased interval in the P2 component of S2. During expiration, the positive intrathoracic pressure causes decreased blood to return to the right side of the heart. The reduced volume in the right ventricle allows the pulmonic valve to close earlier at the end of ventricular systole, causing P2 to occur earlier, and closer to A2. Monitoring of the breathing cycle can be made by measuring the impedance from the device to the tip of the pacing lead.

In an alternative embodiment of the present invention, at least one body position of the patient is detected and it is determined whether the patient is in at least one predetermined specific body posture. In one embodiment of the present invention, the position detecting means is a back-position sensor arranged to sense when the patient is lying on his/her back (or on his or her face). The body posture influences the timing of A2 and P2 in a similar way as the breathing cycle. Therefore, the accuracy of the optimization can be increased by measuring the heart sounds and/or performing the optimization when the patient is found to be within the predetermined body posture. Of course, one or more positions can be detected, for example, when the patient is supine (lying down) and when the patient is in an upright position and thus one optimal setting of stimulation parameters can be obtained for the supine position and another setting of stimulation parameters can be obtained for the upright position. The posture of the patient can be made using, for example, a triaxial accelerometer.

In yet another embodiment of the present invention, at least one activity level of the patient is sensed and it is checked or determined whether the activity level is below a predetermined activity level. The optimization is initiated if the activity level of the patient is found to be below predetermined level. Moreover, a sensing session of the acoustic sensor may be synchronized with a determination that the activity level of the patient is below the predetermined level. Alternatively, it is determined or checked whether the sensed activity level is within a activity level range and the optimization is initiated if the activity level of the patient is found to be within the predetermined range. Moreover, a sensing session of the acoustic sensor may be synchronized with a determination that the activity level of the patient is within the predetermined range. Thereby, it is possible to perform the measurements and the optimization at stable conditions. This predetermined activity level can, for example, be set such that an activity level below the predetermined level indicates rest. The activity level information may be used to further enhance the accuracy of the optimization.

In embodiments of the present invention, the acoustic sensor is arranged in a lead connectable to the device and is located e.g. in the right ventricle of the heart of the patient, or in a coronary vein of the patient, for example, on the epicardial surface in the coronary vein. Other locations is also possible, for example, the sensor may be placed in the right atrium or in the left atrium.

According to embodiments of the present invention, the acoustic sensor is an accelerometer, a pressure sensor or a microphone.

In an alternative embodiment of the present invention, the sensor is arranged within the housing of the implantable device.

As realized by the person skilled in the art, the methods of the present invention, as well as preferred embodiments thereof, are suitable to realize as a computer program or a computer readable medium.

The features that characterize the invention, both as to organization and to method of operation, together with further objects and advantages thereof, will be better understood from the following description used in conjunction with the accompanying drawings. It is to be expressly understood that the drawings are for the purpose of illustration and description and is not intended as a definition of the limits of the invention. These and other objects attained, and advantages offered, by the present invention will become more fully apparent as the description that now follows is read in conjunction with the accompanying drawings.

### BRIEF DESCRIPTION OF THE DRAWINGS

In the following detailed description, reference will be made to the accompanying drawings, of which:

FIG. 1 is block diagram of the primary functional components of a first embodiment of the medical device according to the present invention.

FIGS. 2a, 2b, and 2c are block diagrams of embodiments of a signal processing circuit according to the present invention.

FIG. 3 is a block diagram of the primary functional components of another embodiment of the medical device according to the present invention.

FIG. 4 is a flow chart of the principle steps of the method according to the present invention.

FIG. 5a shows a typical cardiac cycle at a heart rate of 75 BPM, related heart sounds, and the resulting signals in a one sensing procedure according to the present invention.

FIG. 5b shows a typical cardiac cycle at a heart rate of 75 BPM, related heart sounds, and the resulting signals in another sensing procedure according to the present invention.

FIG. 5c shows a typical cardiac cycle at a heart rate of 75 BPM, related heart sounds, and the resulting signals in a one sensing procedure according to the present invention.

FIG. 5d shows a typical cardiac cycle at a heart rate of 75 BPM, related heart sounds, and the resulting signals in another sensing procedure according to the present invention.

FIGS. 6a-6d show schematically an approach to the AV and VV interval optimization procedure.

FIG. 7 shows schematically another approach for optimizing the AV and VV interval.

### DETAILED DESCRIPTION OF THE INVENTION

In the following, the present invention will be discussed in the context of a CRT (Cardiac Resynchronization Therapy) pacemaker. The present invention may also be implemented in other devices such as CRT defibrillators.

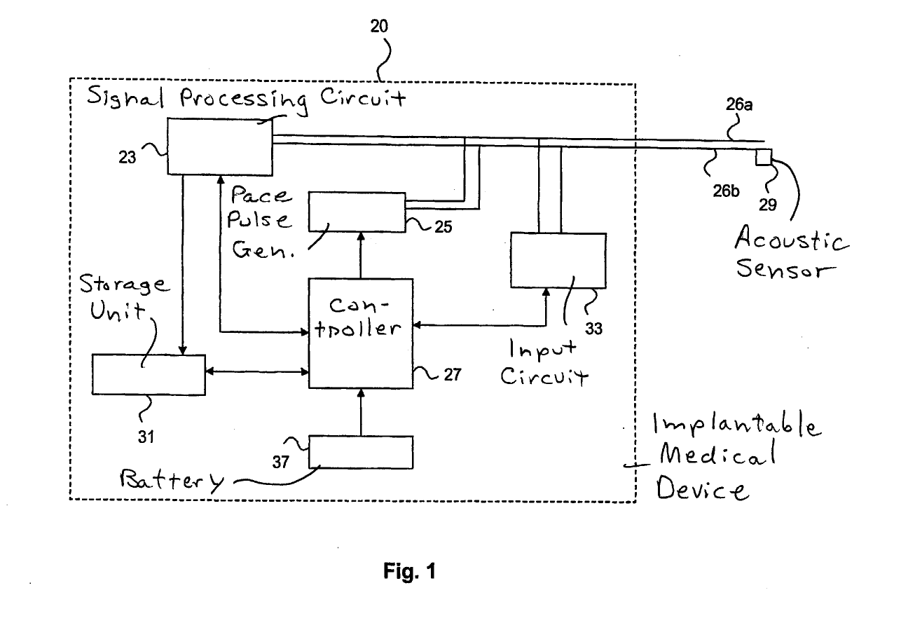

With reference first to FIG. 1, the configuration including the primary components of an embodiment of the present invention will be described. The illustrated embodiment comprises an implantable medical device 20, such as a CRT pacemaker. The pacemaker 20 pacemaker comprises a housing (not shown) being hermetically sealed and biologically inert. Normally, the housing is conductive and may, thus, serve as an electrode. The pacemaker 20 is connectable to one or more pacemaker leads, where only two are shown in FIG. 1 namely a right ventricular lead 26a and a right atrial lead 26b. The leads 26a and 26b can be electrically coupled to the pacemaker 20 in a conventional manner. The leads 26a, 26b extend into the heart (not shown) via a vein of the patient. One or more conductive electrodes for receiving electrical cardiac signals and/or for delivering electrical pacing to the heart are arranged near the distal ends of the leads 26a, 26b. As the skilled man in the art realizes, the leads 26a, 26b may be implanted with its distal end located in either the atrium or ventricle of the heart, or in the coronary sinus or in the great cardiac vein, or they may be in form of epicardial leads attached directly at the epicardium

The leads 26a, 26b may be unipolar or bipolar, and may include any of the passive or active fixation means known in the art for fixation of the lead to the cardiac tissue. As an example, the lead distal tip (not shown) may include a tined tip or a fixation helix. The leads 26a, 26b comprises one or more electrodes, such as a tip electrode or a ring electrode, arranged to, inter alia, measure the impedance or transmit pacing pulses for causing depolarization of cardiac tissue adjacent to the electrode(-s) generated by a pace pulse generator 25 under influence of a controller 27 including a microprocessor. The controller 27 controls, inter alia, pace pulse parameters such as output voltage and pulse duration.

Furthermore, an acoustic sensor 29 is arranged in or connected to one of the leads 26a, 26b, connectable to the device. Alternatively, the acoustic sensor can be located within the housing of the device 20. In one embodiment, the acoustic sensor 29 is arranged in a lead located at the left ventricle of the patient, for example, in the coronary vein on the left ventricle. According to examples, the acoustic sensor 29 is an accelerometer, a pressure sensor or a microphone. The acoustic sensor 29 may also be a piezo electric sensor. The acoustic sensor 29 is adapted to sense acoustic energy of the heart and to produce signals indicative of heart sounds of the heart of the patient. For example, the acoustic sensor 29 may sense the acoustic energy over predetermined periods of a cardiac cycle during successive cardiac cycles.

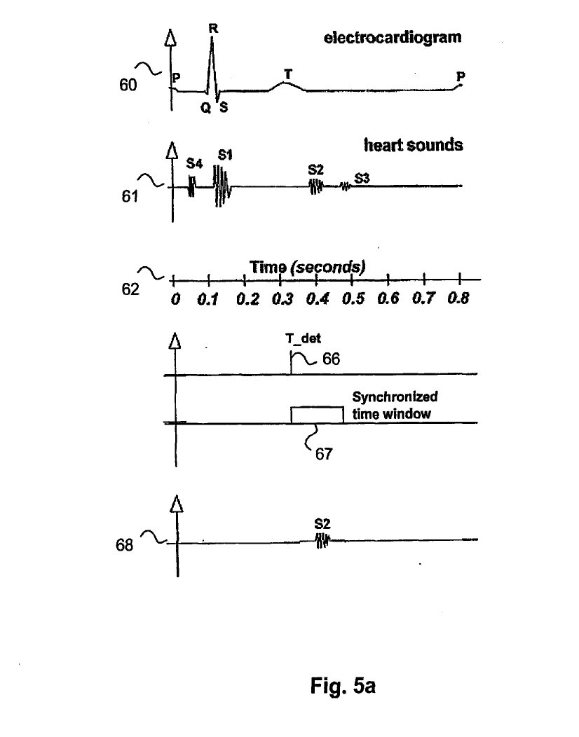

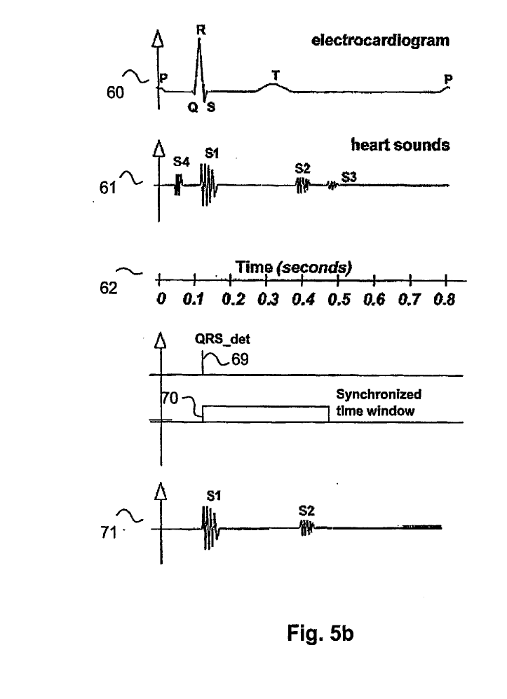

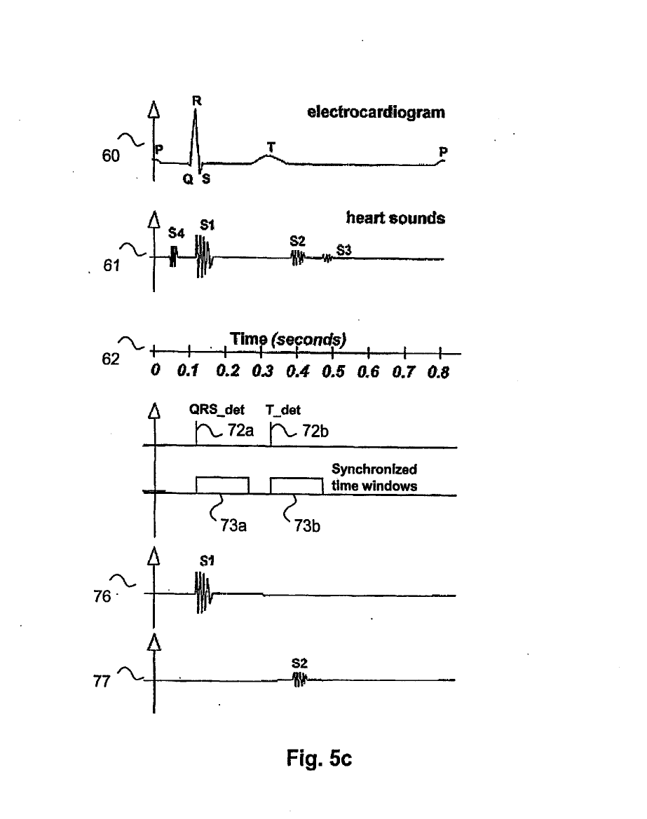

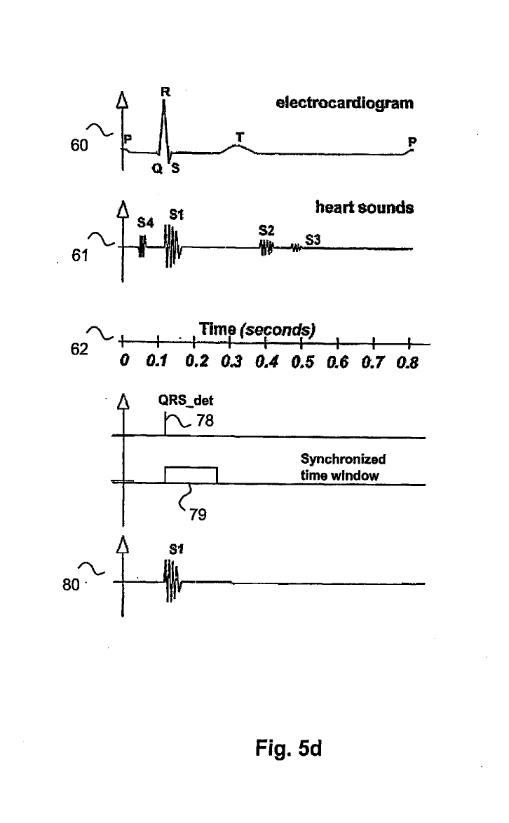

In one embodiment of the present invention, a sensing session to obtain a signal indicative of the second heart sound (S2) is synchronized with a detected heart event, e.g. the detection of an onset (or offset) of an T-wave (see FIG. 5a). The signal is measured during a time window having a predetermined length being synchronized with the detection of the onset (or offset) of the T-wave.

In a further embodiment of the present invention, a sensing session to obtain a signal indicative of a first heart sound (S1) and a second heart sound (S2) is synchronized with a detected heart event, e.g. detection of an intrinsic or paced QRS-complex (see FIG. 5b). Thus, the signal is measured during a time window with a predetermined length being synchronized with the detection of the QRS-complex.

In yet another embodiment, a first sensing session to obtain a first signal indicative of a first heart sound (S1) is synchronized with a detection of an intrinsic or paced QRS-complex and a second sensing session to obtain a second signal indicative of a second heart sound (S2) is synchronized with a detection of an onset (or offset) of an T-wave within the same cardiac cycle (see FIG. 5c). In other words, the signals are measured during two time windows with predetermined lengths, the first being synchronized with the detection of the QRS-complex and the second being synchronized with the detection of the T-wave.

In another embodiment of the present invention, a sensing session to obtain a signal indicative of a first heart sound (S1) is synchronized with a detected heart event, e.g. detection of an intrinsic or paced QRS-complex (see FIG. 5d).

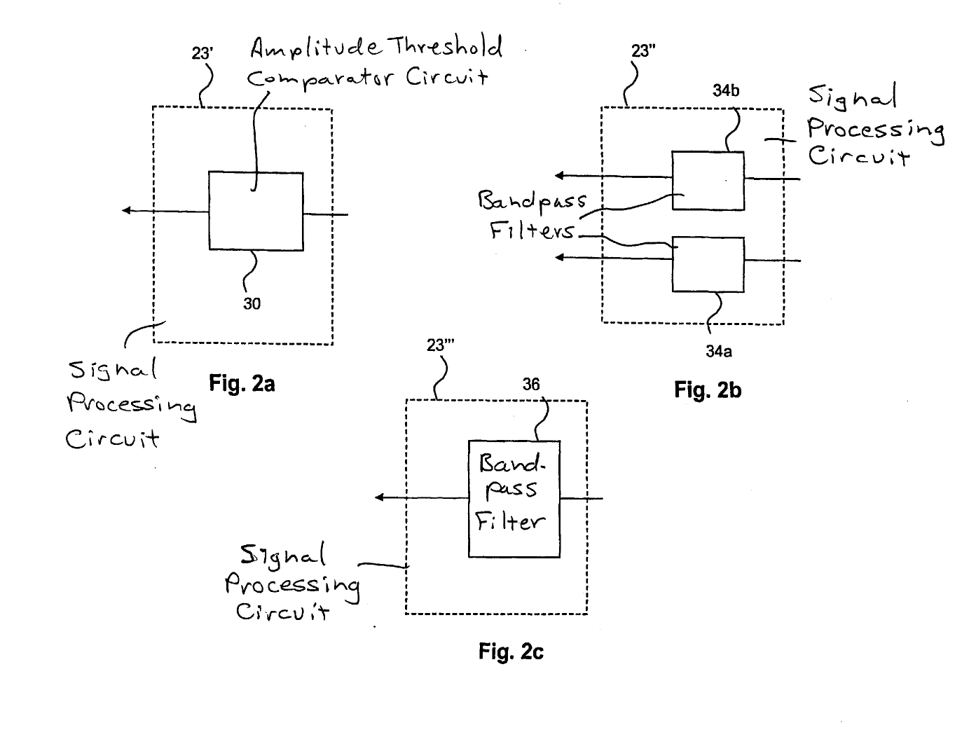

Furthermore, the implantable medical device 20 comprises a signal processing circuit 23 adapted to process sensed signals received from the acoustic sensor 29. Embodiments of the signal processing circuit 23 is shown in FIGS. 2a, 2b and 2c.

According to one embodiment, see FIG. 2a, the signal processing circuit 23′ comprises an amplitude threshold comparator circuit 30 adapted to determine signals corresponding to a first heart sound (S1) and/or a second heart sound (S2) of a sensed signal to be parts of the sensed signal having an amplitude above a predetermined amplitude level.

In another embodiment, see FIG. 2b, the signal processing circuit 23″ comprises pre-process circuits including one or several bandpass filters 34 adapted to filter off frequency components of the sensed signals outside a predetermined frequency range. The bandpass filters may be a digital filter of second order and adapted to perform a zero-phase procedure to cancel out time intervals introduced by the filters. In one embodiment, the signal processing circuit 23″ comprises two bandpass filter 34a and 34b. A first bandpass filter 34a is adapted to cut out a predetermined frequency range corresponding to typical frequencies for the first heart sound, for example, 20-40 Hz, and a second bandpass filter 34b is adapted to cut out a predetermined frequency range corresponding to typical frequencies for the second heart sound, for example, 20-100 Hz, or 10-300 Hz.

In yet another embodiment, see FIG. 2c, the signal processing circuit 23′″ comprises pre-process circuits including one bandpass filter 36 adapted to filter off frequency components of the sensed signals outside a predetermined frequency range. The bandpass filter 36 may be a digital filter of second order and adapted to perform a zero-phase procedure to cancel out time intervals introduced by the filter. The bandpass filter 42 may be adapted to cut out a predetermined frequency range corresponding to typical frequencies for the first and second heart sound, for example, 10-300 Hz.

Returning now to FIG. 1, a storage means 31 is connected to the controller 27, which storage means 31 may include a random access memory (RAM) and/or a non-volatile memory such as a read-only memory (ROM). Storage means 31 is connected to the controller 27 and the signal processing circuit 23. Successive energy values corresponding to a signal corresponding to a first heart sound (S1) and/or to a second heart sound (S2) may for example be stored in the storage means 31.

Detected signals from the patients heart are processed in an input circuit 33 and are forwarded to the controller 27 for use in logic timing determination in known manner. The implantable medical device 20 is powered by a battery 37, which supplies electrical power to all electrical active components of the medical device 20. Data contained in the storage means 31 can be transferred to a programmer (not shown) via a programmer interface (not shown) for use in analyzing system conditions, patient information, etc.

The implantable medical device 20 according to the present invention may also comprise alarm means (not shown) adapted to send an alarm signal indicating that a specific condition has been detected or if a change of a specific condition has been detected. That is, the controller sends a triggering command to the alarm means if a specific condition has been detected or if a change of a specific condition has been detected. The alarm means may be a vibrator causing the device to vibrate or it may be adapted to deliver a beeping sound in order to alert the patient of the situation. Furthermore, an alarm signal can, for example, also or instead be sent to the programmer (not shown) via the programmer interface (not shown). The external unit, i.e. the programmer may be in contact with a central monitoring unit, e.g. at the hospital. In another embodiment, the alarm means is integrated into the controller 27.

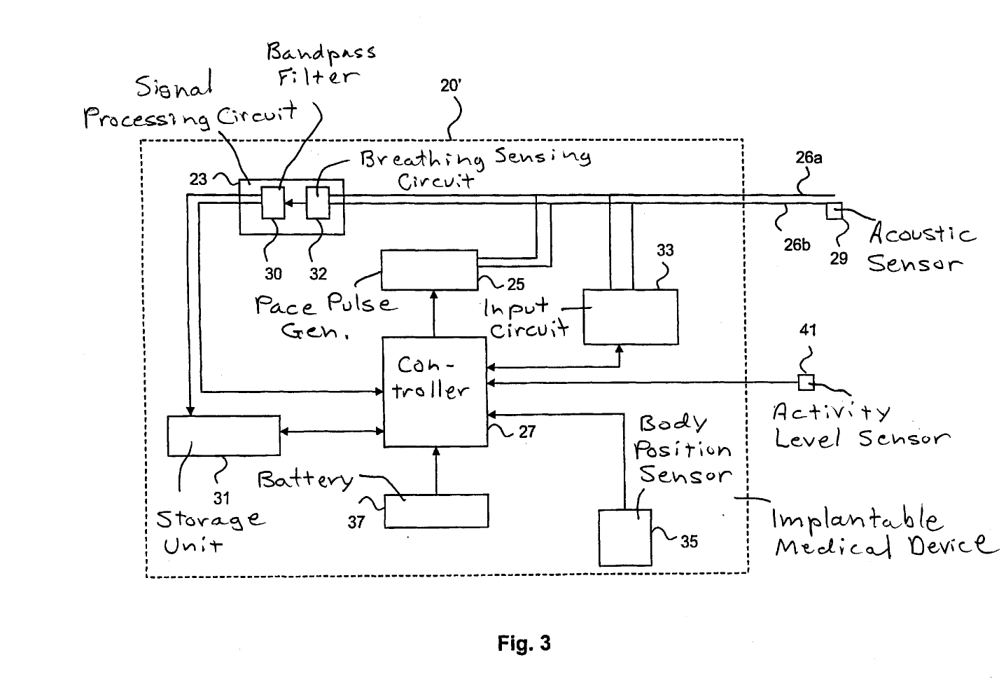

With reference now to FIG. 3, another embodiment of the present invention will be described. Like parts in FIG. 1 and FIG. 3 are denoted with the same reference numeral and the description thereof will be omitted since they have been described with reference to FIG. 1.

The implantable medical device 20′ according to the present invention may comprise a position detecting sensor 35 arranged to detect at least one body position of the patient, for example, a triaxial accelerometer. For example, the position sensor 35 can be adapted to detect a predetermined specific body position. In a one embodiment of the present invention, the position detecting sensor is a back-position sensor arranged to sense when the patient is lying on his/hers back (or on his or hers face). The position detecting sensor 35 is connected to the controller 27. The controller 27 may be adapted to determine whether the patient is in the at least one predetermined specific body position and to synchronize sensing sessions of the acoustic sensor 29 with a determination that the patient is in a predetermined position. Moreover, the optimization procedure may be synchronized with the determination that the patient is in the at least one predetermined specific body position.

Further, the implantable medical device 20′ according to the present invention may include a breathing sensing circuit (not shown) for sensing a breathing cycle of the patient, which circuit is connected to the controller 27. This may, for example, be performed by measuring the impedance from the device 20 to the tip of a pacing lead in accordance with practice within the art. The controller 27 may be adapted to synchronize sensing sessions of the acoustic sensor 29 with a certain point in the breathing cycle, for example, inspiration or expiration, or with a determination that a sensed breath rate is within a predetermined breath rate level range, below a predetermined breath rate level or above a predetermined breath rate level. Moreover, the optimization procedure may be synchronized with the determination that a sensed breath rate is within a predetermined breath rate level range, below a predetermined breath rate level or above a predetermined breath rate level.

Furthermore, the implantable medical device 20′ may also include activity level sensing means 41 for sensing an activity level of the patient, which activity level sensing means is connected to the controller 27. The controller 27 may be adapted to determine whether a sensed activity level is below a predetermined activity level The controller 27 may be adapted to synchronize a sensing session of the acoustic sensor 29 with a determination that the sensed activity level is below a predetermined activity level or that the sensed activity level is within a activity level range between a second activity level and a third activity level. Moreover, the optimization procedure may be synchronized with the determination that the patient is, for example, within a specific activity level range.

As the skilled man realizes, only one, some of or all of the following features: the activity level sensing means 41, the breathing sensing circuit, or the position detector 35, may be included in the medical device according to the present invention. Thus, information from one, some of, or all of the above-mentioned sensors may be used in the optimization.

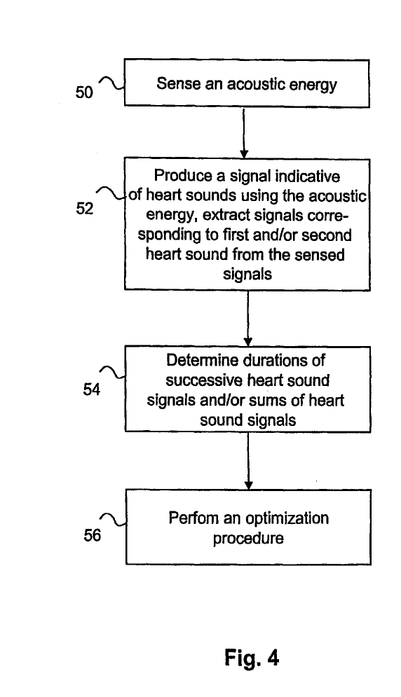

Turning now to FIG. 4, a high-level description of the method according to the present invention will be given. First, at step 50, the acoustic sensor 29 senses an acoustic energy. Then, at step 52, signals indicative of heart sounds of the heart of the patient is produced. This may be performed over predetermined periods of a cardiac cycle during successive cardiac cycles under control of the controller 27. The sensor 29 can be adapted to sense the acoustic energy during predefined time windows in the heart cycle, which will be described hereinafter with reference to FIGS. 5a-5d. In FIGS. 5a-5d, a typical cardiac cycle at a heart rate of 75 beats per minute (bpm), related heart sounds, and the resulting signals in four alternative sensing procedures according to the present invention are shown, respectively.

Referring first to FIG. 5a. A surface electrocardiogram and the related heart sounds S1, S2, S3, and S4 are indicated by 60 and 61, respectively, and a time axis is indicated by 62. In one embodiment, the acoustic sensor 29 is activated by a pacing pulse or the detection of a T-position, as indicated by 66a in FIG. 5a, an intrinsic detected event or a paced event indicated by 60. The acoustic sensor 29 senses the acoustic energy of the heart sound S2, indicated by 61, during a sensing session having a predetermined length, i.e. during predetermined time window, indicated by 67. In this embodiment, the initiation of the sensing session (i.e. the start of the time window) is synchronized with the detection of the T-position indicated by 66. The length of the time window is programmable and a typical length is about 200 ms. Hence, the acoustic sensor 29 receives a triggering signal from the controller 27 upon detection of the T-position by the input circuit 33. The produced signal corresponding to the second heart sound S2 is indicated by 68. This may be performed during successive cardiac cycles under control of the controller 27, which thus produces a time series of successive heart sound signals. The produced signal or signals indicative of the second heart sound are then supplied to the signal processing circuit 23 where, as will be described below in further detail, a signal or signals corresponding to a second heart sound (S2) are extracted from the sensed signal in the signal processing circuit 23 by the pre-processing circuits.

Turning now to FIG. 5b, the same surface electrocardiogram and the related heart sounds S1, S2, S3, and S4 as in FIG. 5a are shown but the sensing procedure is performed in a alternative way. According to this embodiment, the acoustic sensor 29 is activated by a pacing pulse or the detection of a QRS-position, as indicated by 72 in FIG. 5b, an intrinsic detected event or a paced event indicated by 60. The acoustic sensor 29 senses the acoustic energy of the heart sound S1 and of the heart sound S2, indicated by 61, during a sensing session having a predetermined length, i.e. during predetermined time window, indicated by 70. In this embodiment, the initiation of the sensing session (i.e. the start of the time window) is synchronized with the detection of the QRS-position indicated by 69. The length of the time window is programmable and a typical length is about 400 ms. Hence, the acoustic sensor 29 receives a triggering signal from the controller 27 upon detection of the QRS-position by the input circuit 33. The produced signal corresponding to the first heart sound S1 and the second heart sound S2 is indicated by 71. This may be performed during successive cardiac cycles under control of the controller 27, which thus produces a time series of successive heart sound signals. The produced signal or signals indicative of the first heart sound and the second heart sound are then supplied to the signal processing circuit 23 where, as will be described below in further detail, a signal or signals corresponding to a first heart sound (S1) and a second heart sound (S2) are extracted from the sensed signal in the signal processing circuit 23 by the pre-processing circuits.

With reference now to FIG. 5c, the same surface electrocardiogram and the related heart sounds S1, S2, S3, and S4 as in FIGS. 5a and 5b are shown but the sensing procedure is performed in a alternative way. According to this embodiment, the acoustic sensor 29 is activated by a pacing pulse or the detection of a QRS-position and by the detection of a T-position, as indicated by 72a and 72b, respectively, in FIG. 5c, which may be intrinsic detected events or paced events. The acoustic sensor 29 senses the acoustic energy in the heart sound S1, indicated by 61, during a first sensing session or predetermined time window 73a. In this embodiment, the initiation of the first sensing session is synchronized with the detection of the QRS-position. The length of the time window is programmable and a typical length is about 200 ms. Moreover, the acoustic sensor 29 senses the acoustic energy in the heart sound S2, indicated by 61, during a second sensing session or predetermined time window 73b. In this embodiment, the initiation of the second sensing session is synchronized with the detection of the T-position. The length of the time window is programmable and a typical length is about 200 ms. Hence, the acoustic sensor 29 receives a first triggering signal from the controller 27 upon detection of the QRS-position by the input circuit 33 and a second triggering signal from the controller 27 upon the detection of the T-position. The produced signals corresponding to the first heart sound S1 and the second heart sound S2 are indicated by 76 and 77, respectively. This may be performed during successive cardiac cycles under control of the controller 27, which thus produces a time series of successive heart sound signals. The produced signals indicative of the first heart sounds and second heart sounds are then supplied to the signal processing circuit 23 where, as will be described below in further detail, signals corresponding to a first heart sound (S1) and signals corresponding to the second heart sound (S2) are extracted from a sensed signal in the signal processing circuit 23 by the pre-processing circuits.

Referring now to FIG. 5d, the same surface electrocardiogram and the related heart sounds S1, S2, S3, and S4 as in FIGS. 5a, 5b and 5c are shown but the sensing procedure is performed in a alternative way. According to this embodiment, the acoustic sensor 29 is activated by a pacing pulse or the detection of a QRS-position, as indicated by 78 in FIG. 5d, which may be intrinsic detected events or paced events. The acoustic sensor 29 senses the acoustic energy in the first heart sound S1, indicated by 61, during a first sensing session or predetermined time window 79. In this embodiment, the initiation of the first sensing session is synchronized with the detection of the QRS-position. The length of the time window is programmable and a typical length is about 200 ms. Hence, the acoustic sensor 29 receives a triggering signal from the controller 27 upon detection of the QRS-position by the input circuit 33. The produced signal corresponding to the first heart sound S1 is indicated by 80. This may be performed during successive cardiac cycles under control of the controller 27, which thus produces a time series of successive heart sound signals. The produced signals indicative of the first heart sounds are then supplied to the signal processing circuit 23 where, as will be described below in further detail, signals corresponding to a first heart sound (S1) are extracted from a sensed signal in the signal processing circuit 23 by the pre-processing circuits.

Returning now to FIG. 4, the signal or signals indicative of heart sounds are supplied to the signal processing circuit 23 where, at step 52, signals corresponding to a first heart sound (S1) and/or a second heart sound (S2) are extracted from a sensed signal of a cardiac cycle. Optionally, this step may include performing a filtering procedure in order to filter the sensed signal. In one embodiment, a second heart sound signal is determined to be a part of the sensed signal above a predetermined amplitude level and in another embodiment, a first heart sound signal is determined to be a part of the sensed signal having an amplitude above a predetermined amplitude level and a second heart sound signal is determined to be a part of the sensed signal above a second predetermined amplitude level using the amplitude threshold comparator circuit 30, see FIG. 2a. Alternatively, the sensed signals may be bandpass filtered. In one embodiment, two bandpass filters 34a and 34b are used, see FIG. 2b. A first filter 34a adapted to receive a first heart sound signal waveform, see 76 in FIG. 5c, and to cut out a frequency range of 20-40 Hz to form a signal corresponding to the first heart sound (S1) and a second filter 34b adapted to receive a second heart sound signal waveform, see 77 in FIG. 5c, and to cut out a frequency range of 10-300 Hz to form a signal corresponding to the second heart sound (S2). Alternatively, one bandpass filter 36 is used, see FIG. 2c. The filter 42 is adapted to receive a heart sound signal waveform comprising the first heart sound and/or the second heart sound, and to cut out a frequency range of 10-300 Hz to form a signal containing the first heart sound (S1) and/or the second heart sound (S2), see signal waveforms 68, 71, or 80 in FIGS. 5a, 5c, and 5d, respectively. The bandpass filtering process may be performed as a zero-phase procedure to cancel out time intervals introduced by the filters. The sensed signal is in that case in fact filtered twice, first in the forward direction and second in the backward direction.

Returning again to FIG. 4, at step 54, durations of successive heart sound signals and/or sums of heart sound signals are determined or calculated. According to one embodiment, the durations of successive second heart sounds (S2) are determined. Subsequently, at step 56, an optimization procedure is initiated and performed. The optimization procedure will be described below in more detail. In this first embodiment, the pacing pulses are controlled iteratively based on the determined durations of successive second heart sounds to determine a combination of stimulation intervals including at least one of an AV interval and a VV interval that causes a substantially synchronized closure of the aortic and pulmonary valves.

In another embodiment, durations of successive first heart sounds are determined in step 54 and an optimization procedure is initiated at step 56. In this embodiment, the pacing pulses are controlled iteratively based on determined durations of successive first heart sounds to identify stimulation intervals including a VV interval that causes a substantially synchronized closure of the mitral and tricuspid valves.

According to a further embodiment, sums of the durations of first heart sounds and durations of second heart sounds, respectively, for successive cardiac cycles are calculated in step 54. In the optimization procedure, the pacing pulses are controlled iteratively based on the calculated sums of the durations to identify a stimulation interval combination that causes a substantially synchronized closure of the mitral and tricuspid valves and/or the aortic and pulmonary valves, respectively. Thus, the following sum is calculated:

Sum{duration_S1+duration_S2} (1)

In an alternative embodiment, the sum of a duration of a first heart sound and a duration of a second heart sound for successive cardiac cycles is calculated such that the first heart sound is weighted with a first weight, a1, and the second heart sound is weighted with a second weight, a2. Hence, the following sum is calculated:

Sum{a1*duration_S1+a2*duration_S2} (2)

In a further embodiment, the durations of the period of time from start of the first heart sound to the end of the second heart sound for successive heart cycles are calculated. In the optimization procedure, the pacing pulses are controlled iteratively based on the calculated durations of the period of time from start of the first heart sound to the end of the second heart sound to identify a stimulation interval combination that minimizes the systolic phase.

Hereinafter, a number of embodiments of the optimization procedure will be described in detail. The procedures will be described with reference to an optimization of the AV and VV delays with respect to the S2 split. However, as the person skilled within the art realizes, a similar or corresponding procedure may also be applied when optimizing the VV interval with respect to the S1 split, when optimizing the AV and VV intervals with respect to the sum of the S1 and S2 splits, or when optimizing the AV and VV intervals with respect to the period from the start of S1 to the end of S2.

According to a first procedure, all possible combinations of AV and VV intervals within a predetermined interval combination space are evaluated with respect to the S2 split and the combination resulting in the smallest S2 split, i.e. the shortest S2 duration, is selected as setting for the device. Preferably, the AV and VV delays are adjusted step-wise, for example, it may be 10 ms for the AV interval and 5 ms for the VV interval. Moreover, a each combination should be tested a number of times so that an average or median can be calculated.

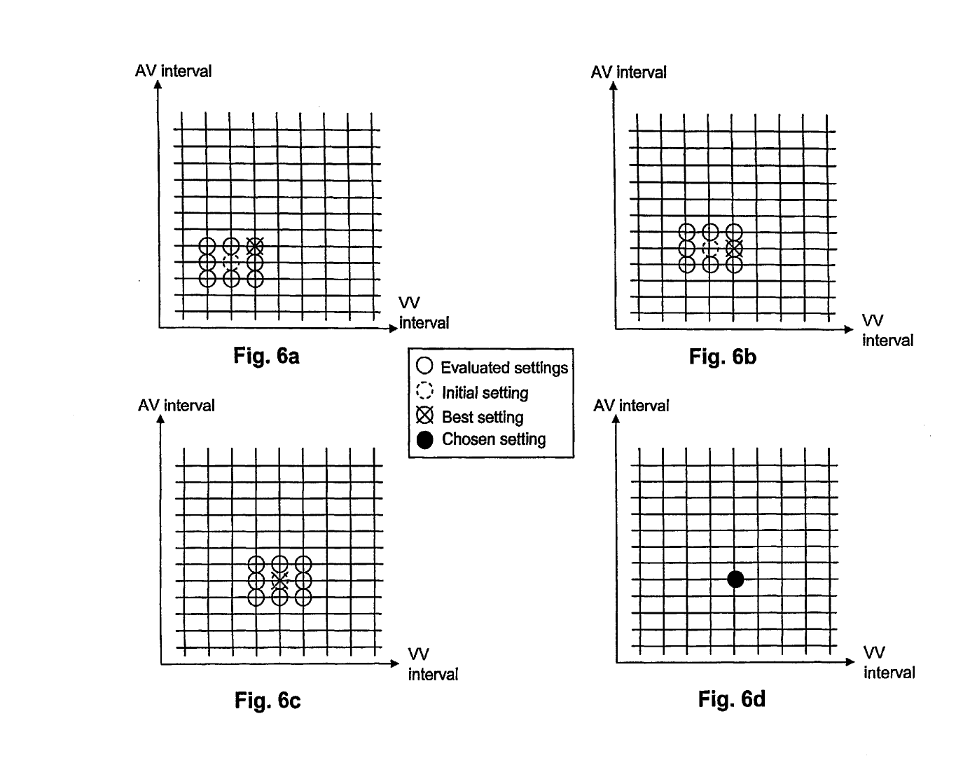

In a second embodiment, the optimization starts with an initial setting of the device or with a setting set by the physician. The initial setting may also be selected at random. In this approach, all adjacent settings are tested and a step size may be 10 ms for AV (delta) and VV (delta2), see FIG. 6a. That is, all combinations of AV, AV+delta, AV-delta, VV, VV+delta2, and VV-delta2 are tested as can be seen in FIG. 6a. The setting with the smallest S2 separation, i.e. the shortest duration, is then chosen as the midpoint and the procedure is repeated, see FIG. 6b. The procedure is repeated until none of the adjacent settings improves the synchronizity, i.e. offer a better value with respect to the S2 separation, see FIG. 6c. The midpoint combination of AV and VV interval is thus selected as setting for the device and the optimization procedure is completed, see FIG. 6d.

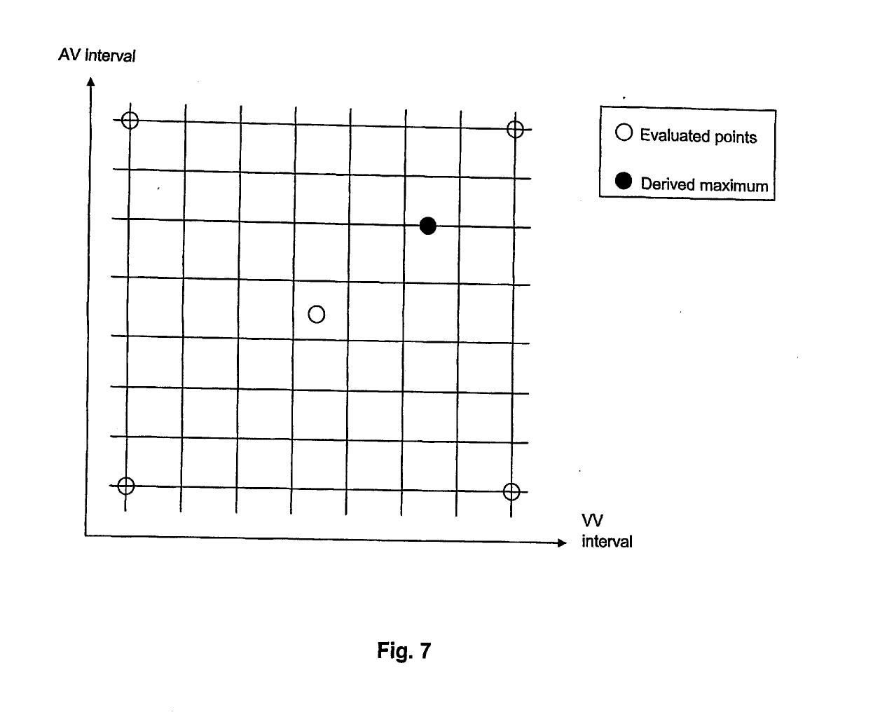

According to a third embodiment, a so called design of experiment approach is used. In this procedure, the boundary combinations of the AV and VV intervals of the predetermined combination space are evaluated together with at least one midpoint value, see FIG. 7. A polynomial that approximates the S2 duration resulting from the different AV and VV intervals is thereafter determined. The maximum value of the polynomial within the combinations space is then derived, i.e. a combination of an AV interval and a VV interval that results in a minimum duration of the second heart sound within the combination space is identified. This AV and VV interval combination may then be selected as setting for the device, see FIG. 7. Alternatively, the combination identified by means of the polynomial may be evaluated. For example, such an evaluation may be performed by testing the identified combination and the adjacent combinations, i.e. the setting are changed one “step” at each direction in accordance with the procedure described above with reference to FIGS. 6a-6d. An example step size may be 10 ms for the AV interval and 5 ms for the VV interval. If the identified combination results in the shortest S2 duration, this combination is selected as setting. If any one the adjacent combinations results in a shorter duration, the procedure is repeated with the new combination as centre combination. This may be repeated until none of the adjacent combinations improve the synchronicity, i.e. until none of the adjacent combinations provide a shorter S2 duration than the centre combination.

The optimization may be performed automatically at regular intervals (i.e. once a day, once a week or once a month), at follow-ups by the request of the physician (i.e. in the hospital) or at the request of the patient (e.g. by the application of a magnet or similar).

Furthermore, in order to improve the efficiency and accuracy of the optimization procedure, information regarding the breathing cycle and/or the patient's body posture can be obtained and used. It has been shown that the S2 duration can depend not only on dyssynchrony, but also on where in the breathing cycle the measurement is made. During inspiration, negative intrathoracic pressure causes increased blood return into the right side of the heart. The increased blood volume in the right ventricle causes the pulmonic valve to stay open longer during ventricular systole. This causes an increased interval in the P2 component of S2, i.e. the closure of the pulmonary valve. During expiration, the positive intrathoracic pressure causes decreased blood return to the right side of the heart. The reduced volume in the right ventricle allows the pulmonic valve to close earlier at the end of ventricular systole, causing P2 to occur earlier and closer to the A2 component of S2, i.e. the closure of the aortic valve. In a corresponding way, body posture can influence the timing of A2 and P2. According to one embodiment, to improve the accuracy of the optimization procedure, the breathing cycle is sensed and the acoustic sensor is triggered to sense the acoustic energy at certain point in the breathing cycle. In another embodiment, the optimization is performed when the patient is in a certain posture. In a further embodiment, the heart sound measurements are made only at a certain point in the breathing cycle and at a certain body posture. As a complement or as an alternative, an average of measurements from a period of, for example, several minutes may be used to further reduce the artifacts.

Although an exemplary embodiment of the present invention has been shown and described, it will be apparent to those having ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims.

I claim as my invention:

1. An implantable medical device
a pulse generator that emits cardiac stimulating pacing pulses at least one lead comprising electrodes for delivering said pulses to cardiac tissue in at least one ventricle of a heart of a patient;
an acoustic sensor that detects acoustic energy in the patient and that emits a sensed signal corresponding thereto;
a signal processing circuit configured to extract a signal corresponding to a second heart sound (S2) from the sensed signal, said signal being received from said acoustic sensor, and to produce signals indicative of second heart sounds of the heart of said patient over predetermined periods of a cardiac cycle during successive cardiac cycles, and to determine a duration of successive second heart sound signals; and
a controller configured to perform an optimization procedure that controls delivery of said pacing pulses based on the determined durations of successive second heart sound signals to identify a combination of stimulation intervals including at least one of a AV interval and a VV interval that causes a substantially synchronized closure of the aortic and pulmonary valves.

2. The implantable medical device according to claim 1, wherein said controller is configured to control said stimulation intervals such that the durations of said second heart sound signals are within a predetermined range of durations.

3. The implantable medical device according to claim 1, wherein said controller is configured to control said stimulation intervals such that the durations of said heart sound signals are minimized.

4. The implantable medical device according to claim 1, wherein:
said signal processing circuit is configured to also extract a signal corresponding to a first heart sound (S1) from said sensed signal, to produce signals indicative of first heart sounds of the heart of said patient over predetermined periods of a cardiac cycle during successive cardiac cycles, and to determine a duration of successive first heart sound signals; and
said controller is configured to control delivery of said pacing pulses based on determined durations of successive first heart sound signals to identify a VV interval that causes a substantially synchronized closure of the mitral and tricuspid valves.

5. The implantable medical device according to claim 4, wherein said controller is configured to control the VV interval such that the durations of said first heart sound signals are within a predetermined range of durations.

6. The implantable medical device according to claim 4, wherein said controller is configured to control the VV interval such that the durations of said first heart sound signals are minimized.

7. The implantable medical device according to claim 4, wherein said controller is adapted to:
calculate a sum of a duration of a first heart sound and a duration of a second heart sound for successive cardiac cycles; and
control said pacing pulses based on calculated sums of durations to identify a stimulation interval combination that causes a substantially synchronized closure of the mitral and tricuspid valves and/or a substantially synchronized closure of the aortic and pulmonary valves, respectively.

8. The implantable medical device according to claim 7, wherein said controller is configured to:
control said stimulation interval combination such that said sums of a first heart sound and second heart sound are within a predetermined range of duration sums.

9. The implantable medical device according to claim 7, wherein said controller is configured to:
control said stimulation interval combination such that said sums of a first heart sound and second heart sound are minimized.

10. The implantable medical device according to claim 4, with said controller is configured to:
calculate a sum of duration of the first heart sound and the duration of a second heart sound for successive cardiac cycles, with said first heart sound weighted with a first weight and said second heart sound weighted with a second weight.

11. The implantable medical device according to claim 5, wherein said controller is configured to:
calculate the duration of the period of time from start of the first heart sound to the end of the second heart sound for successive heart cycles; and
control said pacing pulses based on said calculated durations of the period of time from start of the first heart sound to the end of the second heart sound to identify a stimulation interval combination that minimizes the systolic phase.

12. The implantable medical device according to claim 11, wherein said controller is configured to:
control said stimulation interval combinations such that the durations of the period of time from start of the first heart sound to the end of the second heart sound are within a predetermined range of durations.

13. The implantable medical device according to claim 12, wherein said controller is configured to:
control said stimulation interval combinations such that the durations of the period of time from start of the first heart sound to the end of the second heart sound are minimized.

14. The implantable medical device according to claim 1, wherein said stimulation interval includes AV and VV intervals and wherein said controller is configured to:
apply selected combinations of AV and VV intervals within at least one predetermined space of possible interval combinations;
evaluate the durations corresponding to the second heart sound resulting from the selected combinations of AV and VV intervals within said predetermined space of possible interval combinations; and
select the combination of AV and VV intervals that results in a minimized duration of the second heart sound as a setting for controlling said pacing pulse.

15. The implantable medical device according to claim 14, wherein the selected combinations are at least the boundary conditions of said combination space and a midpoint combination of said combination space, wherein said controller is configured to:
determine a polynomial using the evaluated durations resulting from the selected combinations that approximates the resulting durations.

16. The implantable medical device according to claim 15, wherein said controller is configured to:
identify a combination of an AV interval and a VV interval that results in a minimum duration of said second heart sound within said combination space using said polynomial.

17. The implantable medical device according to claim 16, wherein said controller is configured to:
select the identified combination of AV and VV intervals as a setting for controlling deliver of said pacing pulses.

18. The implantable medical device according to claim 17, wherein said controller is configured to:
apply the selected combination of AV and VV interval; and
evaluate the duration corresponding to the second heart sound resulting from the identified combination of AV and VV intervals.

19. The implantable medical device according to claim 1 wherein said stimulation interval includes AV intervals and VV intervals, and wherein said controller is configured to:
a) select an initial combination of an AV interval and a VV interval;
b) define a combination space surrounding said initial combination of AV interval and VV interval;
c) apply each combination of AV and VV intervals in said combination space;
d) evaluate the durations corresponding to the second heart sound resulting from the combinations of AV and VV intervals in said first combination space;
e) identify a minimum duration within said first combination space;
f) set the combination of AV and VV interval resulting in said identified minimum pulse as said initial combination;
g) repeat steps a)-e); and
h) perform a comparison step in order to determine whether a minimum duration has been obtained.

20. The implantable medical device according to claim 19, wherein said wherein said controller is configured to:
if the minimum duration identified in the current combination space is shorter than the preceding identified minimum duration, select the combination of AV and VV intervals resulting in the minimum duration of the current combination space as a setting for controlling delivery of said pacing pulses.

21. The implantable medical device according to claim 19, wherein said controller is configured to:
if the minimum duration identified in the current combination space is longer than or substantially equal to the preceding identified minimum duration, repeat steps a)-h).

22. The implantable medical device according to claim 1, wherein said controller is configured to:
calculate each duration as a mean value over a predetermined number of successive durations or during a predetermined period of time.

23. The implantable medical device according to claim 4, wherein said signal processing circuit comprises:
a first bandpass filter that filters off frequency components of the sensed signals from said acoustic sensor outside a first predetermined frequency range for said second heart sounds to extract said signal corresponding to a first heard sound.

24. The implantable medical device according to claim 4, wherein said signal processing circuit comprises a second bandpass filter that filters off frequency components of said sensed signals from said acoustic sensor outside a second predetermined frequency range for said first heart sounds to extract said signal corresponding to a second heart sound.

25. The implantable medical device according to claim 4, wherein said signal processing circuit comprises:
a bandpass filter that filters off frequency components of said sensed signals outside a predetermined frequency range for said first heart sounds and for said second heart sounds.

26. The implantable medical device according to claim 4, wherein said signal processing circuit is configured to calculate the durations based on a part of the sensed signals above a first predetermined amplitude threshold to produce said signals indicative of said first heart sound and a second predetermined amplitude level to produce said signals indicative of said second heart sound.

27. The implantable medical device according to claim 1, further comprising;
a position detector that detects at least one position of said patient; and said controller is configured to determine whether said patient is in said at least one predetermined specific body position and to initiate said optimization procedure only if said patient is in said predetermined specific body position.

28. The implantable medical device according to claim 1, further comprising:
an activity level sensor that senses an activity level of said patient; and said controller is configured to determine whether said activity level is within a predetermined activity level range and to initiate said optimization procedure only if said sensed activity level is determined to be within said predetermined activity level range.

29. The implantable medical device according to claim 1, further comprising:
a breathing sensing circuit that senses a breathing cycle of said patient; and said controller is configured to identify at least one predetermined point in said breathing cycle of said patient and to synchronize sensing sessions of said acoustic sensor with said at least one predetermined point in said breathing cycle of said patient for successive breathing cycles.

30. The implantable medical device according to claim 1, wherein said acoustic sensor is arranged in a lead electrically connectable to said signal processing circuit.

31. The implantable medical device according claim 30, wherein said lead is configured to locate said acoustic sensor at a site selected from the group consisting of in the right ventricle of the heart of said patient, in the left atrium, in a coronary vein, vena cava, on the epicardium, and in the thorax.

32. The implantable medical device according to claim 1 comprising a device housing, and wherein said acoustic sensor is located within the device housing.

33. The implantable medical device according to claim 1, wherein said acoustic sensor is a sensor selected from the group consisting of accelerometers, pressure sensors and microphones.

34. A method for operating an implantable medical device, said device including a pulse generator adapted to produce cardiac stimulating pacing pulses and being connectable to at least one lead comprising electrodes for delivering said pulses to cardiac tissue, comprising the steps of:
sensing an acoustic energy;
producing signals indicative of heart sounds of the heart of said patient over predetermined periods of a cardiac cycle during successive cardiac cycles;
extracting a signal corresponding to a second heart sound (S2) from a sensed signal;
determining durations of successive second heart sound signals; and
performing an optimization procedure, said optimization procedure comprising the step of controlling said pacing pulses based on said determined durations of successive second heart sounds to determine a combination of stimulation intervals including at least one of an AV interval and a VV interval that causes a substantially synchronized closure of the aortic and pulmonary valves.

35. The method according to claim 34, wherein said optimization procedure comprises the step of controlling the stimulation intervals such that the durations of said second heart sound signals are within a predetermined range of durations.

36. The method according to claim 34, wherein said optimization procedure comprises the step of controlling the stimulation intervals such that the durations of said second heart sound signals are minimized.

37. The method according to claim 34, further comprising the steps of:
extracting a signal corresponding to a first heart sound (S1) from a sensed signal;
determining durations of successive first heart sound signals; and
wherein said optimization procedure further comprises the step of controlling said pacing pulses based on determined durations of successive first heart sounds to identify stimulation interval including a VV interval that causes a substantially synchronized closure of the mitral and tricuspid valves.

38. The method according to claim 37, wherein said optimization procedure comprises the step of controlling the VV interval such that the durations of said first heart sound signals are within a predetermined range of durations.

39. The method according to claim 37, wherein said optimization procedure comprises the step of controlling the VV interval such that the durations of said first heart sound signals are minimized.

40. The method according to claim 37, wherein said optimization procedure further comprises the steps of:
calculating a sum of a duration of a first heart sound and a duration of a second heart sound for successive cardiac cycles; and
controlling said pacing pulses based on calculated sums of durations to identify a stimulation interval combination that causes a substantially synchronized closure of the mitral and tricuspid valves and/or a substantially synchronized closure of the aortic and pulmonary valves, respectively.

41. The method according to claim 40, wherein said step of optimizing further comprises the step of
controlling said stimulation interval combination such that said sums of a first heart sound and second heart sound are within a predetermined range of duration sums.

42. The method according to claim 40, wherein said step of optimizing further comprises the step of controlling said stimulation interval combination such that said sums of a first heart sound and second heart sound are minimized.

43. The method according to claim 37, wherein said optimization procedure further comprises the step of:
calculating a sum of a duration of the first heart sound and a duration of the second heart sound for successive cardiac cycles, and weighting said first heart sound with a first weight and weighting said second heart sound with a second weight.

44. The method according to claim 37, wherein said optimization procedure further comprises the steps of:
calculating the duration of the period of time from start of the first heart sound to the end of the second heart sound for successive heart cycles;
and controlling said pacing pulses based on said calculated durations of the period of time from start of the first heart sound to the end of the second heart sound to identify a stimulation interval combination that minimizes the systolic phase.

45. The method according to claim 44, wherein said optimization procedure comprises the steps of:
controlling said stimulation interval combinations such that the durations of the period of time from start of the first heart sound to the end of the second heart sound are within a predetermined range of durations.

46. The method according to claim 44, wherein said optimization procedure comprises the step of:
controlling said stimulation interval combinations such that the durations of the period of time from start of the first heart sound to the end of the second heart sound are minimized.

47. The method according to claim 34, wherein said stimulation intervals includes AV and VV intervals and wherein said optimization procedure comprises the steps of:
applying selected combinations of AV and VV intervals within at least one predetermined space of possible interval combinations;
evaluating the durations corresponding to the second heart sound resulting from the selected combinations of AV and VV intervals within said predetermined space of possible interval combinations; and
selecting the combination of AV and VV intervals that results in a minimized duration of the second heart sound as setting for said device.

48. The method according to claim 47, wherein the selected combinations are the boundary conditions of said combination space and a midpoint combination of said combination space, further comprising the steps of:
determining a polynomial using the evaluated pulse widths resulting from the selected combinations that approximates the resulting durations.

49. The method according to claim 48, further comprising the step of:
identifying a combination of an AV interval and an VV interval that results in a minimum duration of said second heart sound within said combination space using said polynomial.

50. The method according to claim 49, further comprising the step of:
selecting the identified combination of AV and VV intervals as setting for said device.

51. The method according to claim 49, further comprising the step of:
applying the selected combination of AV and VV interval; and
evaluating the duration corresponding to the second heart sound resulting from the identified combination of AV and VV intervals.

52. The method according to claim 34, wherein said optimization comprises the steps of:
a) selecting an initial combination of an AV interval and a VV interval;
b) defining a combination space surrounding said initial combination of AV interval and VV interval;
c) applying each combination of AV and VV intervals in said combination space;
d) evaluating the durations corresponding to the second heart sound resulting from the combinations of AV and VV intervals in said first combination space;
e) identifying a minimum duration within said first combination space;
f) setting the combination of AV and VV interval resulting in said identified minimum pulse as said initial combination;
g) repeating the steps a)-e);
h) performing a comparison step in order to determine whether a minimum duration has been obtained.

53. The method according to claim 52, wherein said comparison step comprises the step of:
if the minimum duration identified in the current combination space is shorter than the preceding identified minimum duration, selecting the combination of AV and VV intervals resulting in the minimum duration of the current combination space as setting for said device.

54. The method according to claim 52, wherein said comparison step comprises the step of:
if the minimum duration identified in the current combination space is longer than or substantially equal to the preceding identified minimum duration, repeating steps a)-h).

55. The method according to claim 34, wherein the step of determining durations comprises the step of calculating each duration as a mean value over a predetermined number of successive durations or during a predetermined period of time.

56. The method according to claim 37, wherein the step of determining durations of successive first heart sound signals, further comprises the step of:
filtering off frequency components of said sensed signals outside a first frequency range for said second heart sounds.

57. The method according to claim 56, wherein the step of determining durations of successive second heart sound signals, further comprises the step of:
filtering off frequency components of said sensed signals outside a second frequency range for said first heart sounds.

58. The method according to claim 37, wherein the step of determining durations of successive first and second heart sound signals, respectively, further comprises the step of:
filtering off frequency components of said sensed signals outside a predetermined frequency range for said first heart sounds and for said second heart sounds.

59. The method according to claim 37, further comprising the step of calculating the durations based on a part of the signals above a first predetermined amplitude threshold for said first heart sound signals and a second predetermined amplitude level for said second heart sound signals.

60. The method according to claim 34, further comprising the steps of:
detecting a body position of said patient;
determining whether said patient is in a predetermined specific body position; and
only if said patient is in said predetermined specific body position, initiating said optimization procedure.

61. The method according to claim 34, further comprising the steps of:
sensing an activity level of said patient;
determining whether said activity level is within a predetermined activity level range; and
only if said sensed activity level is determined to be within said predetermined activity level range, initiating said optimization procedure.

62. The method according to claim 34, further comprising the steps of:
sensing a breathing cycle of said patient;
identifying at least one predetermined point in said breathing cycle of said patient; and
synchronizing sensing sessions of said acoustic sensor with said at least one predetermined point in said breathing cycle of said patient for successive breathing cycles.

63. The method according to claim 34, comprising carrying said acoustic sensor in a lead connectable to said device.

64. The method according claim 63, comprising placing said acoustic sensor carried in said lead at a site selected from the group consisting of the right ventricle of the heart of said patient, in the left atrium, in a coronary vein, vena cava, on the epicardium, and in the thorax.

65. The method according to claim 34, comprising mounting said acoustic sensor within a housing of said device.

66. The method according to claim 34, comprising selecting said acoustic sensor from the group consisting of accelerometers, pressure sensors and microphones.

67-68. (canceled)

69. A computer-readable medium encoded with programming instructions for use in an implantable medical device, said device including a pulse generator that emits cardiac stimulating pacing pulses and at least one lead connected to the pulse generator comprising electrodes for delivering said pulses to cardiac tissue, and an acoustic energy sensor, said programming instructions causing said implantable medical device to:
sense acoustic energy with said acoustic energy sensor;
produce signals indicative of heart sounds of the heart of the patient over predetermined periods of a cardiac cycle during successive cardiac cycles;
extract a signal corresponding to a second heart sound (S2) from the sensed signal from said acoustic energy sensor;
determine durations of successive second heart sound signals; and
perform an optimization procedure including controlling delivery of said pacing pulses dependent on the determined durations of successive second heart sounds to determine a combination of stimulation intervals, including at least one of an AV interval and VV interval, that causes a substantially synchronized closure of the aortic and pulmonary valves.
